(19) United States
(12) Patent Application Publication
(10) Pub. No.: US 2021/0321897 A1
(43) Pub. Date: Oct. 21, 2021

(54) METHOD FOR REMOTE COLLECTION AND GROUP PROCESSING OF PSYCHOPHYSIOLOGICAL REACTIONS UPON PRESENTATION OF VARIOUS INFORMATION

(71) Applicant: CELEBRIUM LABS LTD., Limassol (CY)

(72) Inventor: Alexey V. IVANOV, Moscow (RU)

(73) Assignee: CELEBRIUM LABS LTD., Limassol (CY)

(21) Appl. No.: 16/850,715

(22) Filed: Apr. 16, 2020

(51) Int. Cl. A61B 5/0484; A61B 5/16; A61B 5/11; A61B 5/00; G06F 3/01

(52) U.S. Cl. CPC A61B 5/0484; A61B 5/162; G06F 3/015; A61B 5/0006; A61B 5/4064; A61B 5/1124

(57) ABSTRACT

Disclosed relates to medicine and, more particularly, to processing of data for special applications. Information is presented in text, audio, or video form. A personal human reaction to the presented information is recorded by determining the motor activity or EEG biopotentials. Based on recording of personal reactions, the obtained data is processed. At the first stage, differences between the stimuli of reference categories are calculated using the relevant statistical difference criterion for each of the analyzed primary indexes in each personal set of indexes. Then, the obtained values reflecting the discriminant ability of each primary index are subjected to factor analysis separately for amplitude and time indexes. The values of obtained factors are calculated. Following this, the respondents are combined into groups based on that which factor had the maximum value. At the second processing stage, estimation of text, audio, and video stimuli are obtained using a set of criteria obtained at the previous stage. The differences between the stimuli of different categories are calculated for each primary index. Then, based on these values and the factor coefficients obtained at the first stage, the values of factors grouping the primary indexes are calculated. The degree of class membership of one of the reference estimations in each group formed at the first stage is calculated for each stimulus in a space of primary indexes with the use of discriminant functions. The obtained classification results are weighed using the value of factor by which this group has been built. The weighed classification results for each group of indexes are summed up. The method allows providing remote collection and group processing of personal psychophysiological reactions of motor activity and neurophysiological indexes of human brain functions, and formation of group user data arrays that make it possible, after processing and due to their volume, to obtain the group indexes of attitude to the information associated with the recorded reactions.

### BACKGROUND OF THE INVENTION

The invention relates to devices or methods for digital calculations or data processing for special applications.

There are known methods for recording personal characteristics of a computer user based on measuring physiologically conditioned features of his/her work on a computer keyboard, for example, by calculating the time intervals between keystrokes. Thus, for example, the US 20080214903 A1 document (published on Apr. 9, 2008) discloses a system and a method for monitoring of one or more user's physiological parameters. The system of the invention includes one or more wearable sensor modules that determine one or more physiological parameters.

### SUMMARY OF THE INVENTION

The technical problem to be solved consists in obtaining group statistical data on the nature of response to presented information in the form of images, text, video, etc. In so doing, objectively recorded reactions such as an electroencephalogram, motor activity indexes are estimated.

The technical result of the claimed invention is automated statistical processing of personal psychophysiological reactions in users, related to presentation of information, and formation of group statistical estimations that characterize the information being presented.

### BRIEF DESCRIPTION OF THE INVENTION

The claimed technical problem is solved, and the technical result is achieved by means of the claimed method for acquisition and processing of data on motor activity and neurophysiological indexes of human brain functions upon presentation of information in the form of text, audio, video, etc., wherein the method allows collecting and analyzing this data remotely for large groups of users and, based on recorded personal reactions, forming group user data arrays that, after processing and due to their volume, make it possible to obtain the group indexes of attitude to the information associated with the recorded reactions, and consists in calculating differences between the stimuli of reference categories (using the relevant statistical difference criterion) for each of the analyzed primary indexes in each personal set of indexes at the first stage, and then subjecting the obtained values reflecting the discriminant ability of each primary index to factor analysis separately for amplitude and time indexes for each sensor and calculating the values of obtained factors, and, subsequently, combining the respondents into groups based on that which of the factors had the maximum value, wherein the second stage of processing consists in obtaining estimations of stimuli using a criteria system obtained at the previous stage, while the processing algorithm of a separate procedure comprises the steps of:

a) calculating differences between the stimuli of different categories for each primary index,

b) then, based on these values and the factor coefficients obtained at the first stage, calculating the values of factors which grouping the primary indexes,

c) for each stimulus in a space of primary indexes with the use of discriminant functions calculating the degree of class membership of one of the reference estimations in each group formed at the first stage,

d) weighing the obtained classification results using the value of factor by which this group has been built on the basis of results of item b,

e) summing up the weighed classification results for each group of indexes.

Personal psychophysiological reactions are recorded with personal devices belonging to the smartphone or tablet computer class, and also using a personal device for recording of biopotentials of human brain (EEG). Presentation of information during the recording is performed by means of personal devices belonging to the class of mobile phone, tablet or desktop computer, or laptop computer, as well as by other tools that ensure presentation of information to a user via different sensory channels such as visual, acoustic, tactile, etc.

Information (content) in the form of images, text, video, etc. is loaded to a server. Preliminary preparation for presentation of content in the form of scenario composed of a set of fragments taking into account their number and exposure time is carried out on the server. The prepared scenario is sent to a personal device. The user launches the scenario presentation program that records indexes of his/her motor (motion) activity during the presentation. Upon completion of scenario presentation, the data on motor activity is sent to the server. The server processes this users' data and forms group statistical estimations that characterize the information (content) being presented.

The obtained technical result contains a combination of two directions:

to obtain group statistical estimations, the results of remote monitoring of large groups of users are employed;

group statistical estimations are based on physiological indexes recording.

### BRIEF DESCRIPTION OF THE DRAWINGS

The patent or application file contains at least one color drawing. Copies of this patent or patent application publication with color drawing will be provided by the USPTO upon request and payment of the necessary fee.

FIG. 1 is a flow diagram of structural components and their interaction during implementation of the method.

FIG. 2 is a flow diagram showing module operation for presentation of information and recording of indexes in a personal device.

FIG. 3 is a processing scheme for personal EEG reactions.

FIG. 4 shows the variability of physiological responses by example of recording of motor reactions upon presentation of various information.

### DETAILED DESCRIPTION OF THE DRAWINGS

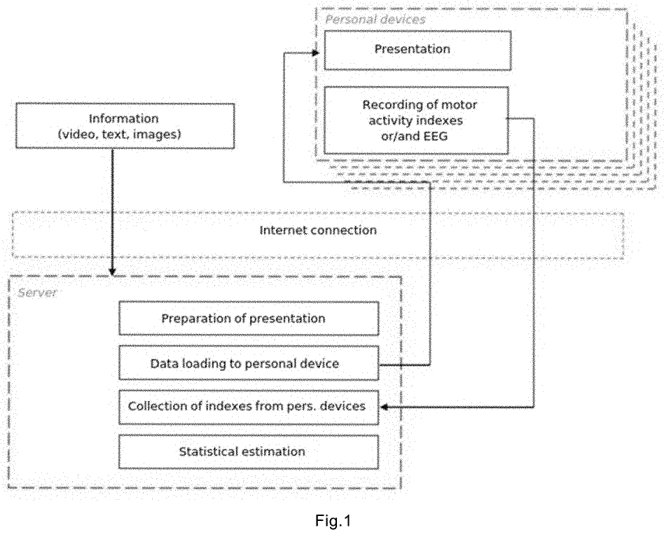

A flow diagram of structural components and their interaction during implementation of the method is shown in FIG. 1.

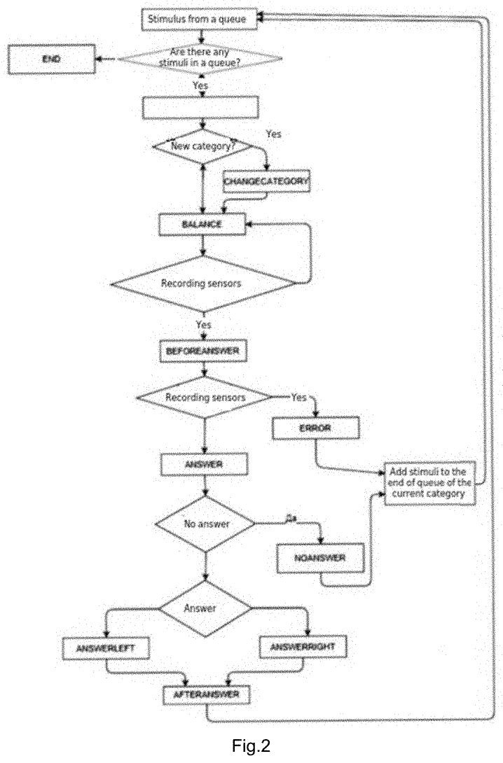

A flow diagram showing module operation for presentation of information and recording of indexes in a personal device is shown in FIG. 2.

Reactions recorded by the personal device of each respondent are processed as follows. At the first stage, differences between the stimuli of reference categories are calculated (using the relevant statistical difference criterion) for each of the analyzed primary indexes in each personal set of indexes. Then, the obtained values reflecting the discriminant ability of each primary index are subjected to factor analysis separately for amplitude and time indexes (principal component analysis, varimax rotation) for each sensor, and the values of obtained factors are calculated. Following this, the respondents are combined into groups based on that which of the factors had the maximum value.

The second processing stage consists in obtaining estimations of stimuli using the criteria system obtained at the previous stage. The processing algorithm of separate procedure can be presented as follows.

a) In a way analogous to what has been done at the first stage, the differences between the stimuli of different categories are calculated for each primary index.

b) Then, based on these values and the factor coefficients obtained at the first stage, the values of factors which grouping the primary indexes are calculated.

c) The degree of class membership of one of the reference estimations in each group formed at the first stage is calculated for each stimulus in a space of primary indexes with the use of discriminant functions.

d) The obtained classification results are weighed using the value of factor by which this group has been built on the basis of results of item b.

e) The weighed classification results for each group of indexes are summed up.

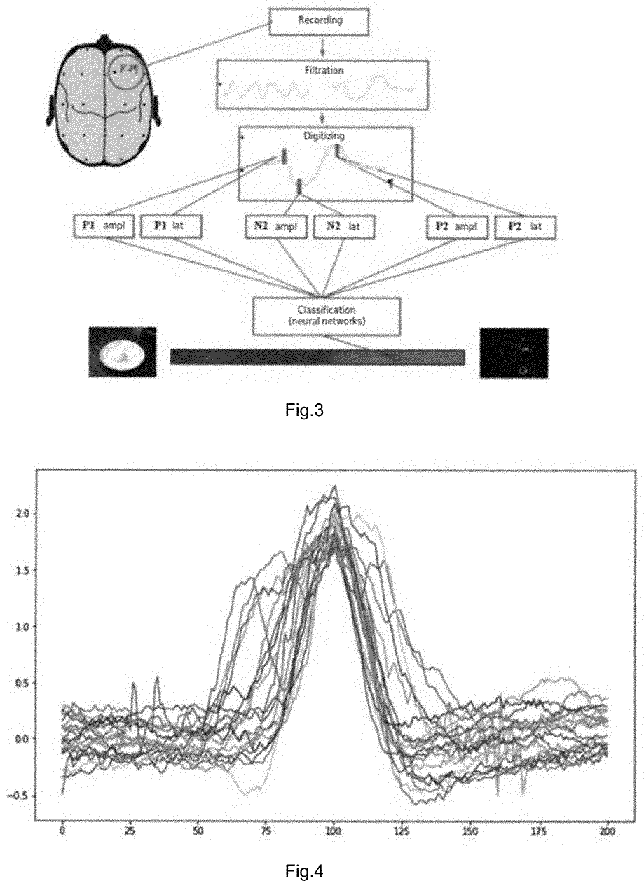

A processing scheme for personal EEG reactions is shown in FIG. 3.

A processing scheme for a single evoked potential by example of a right frontal lead (F4) is presented. P1, P2, N2 are components of the evoked potential, Ampl is the amplitude values, Lat is the latency values.

The same processing algorithm is used for all other EEG leads for all respondents.

Data accumulated in this manner is processed on the server through a multivariate index analysis received from different sensors of personal device that reflect the characteristics of physiological reaction arising in response to presentation of information.

The variability of physiological responses by example of recording of motor reactions upon presentation of various information is given in FIG. 4.

The variability of physiological responses is determined by the content of presented information. The variability analysis for a group of respondents allows estimate the information in terms of its significance concerning to the selected estimation criteria.

1. A method for remote collection and group processing of psychophysiological reactions of motor activity and neurophysiological indexes of human brain functions, comprising presentation of information in text, audio or video form, then recording of a personal human reaction to the presented information by determining the motor activity or EEG biopotentials and, based on the recording of personal reactions, processing of the obtained data, namely:
at the first stage, calculating differences between the stimuli of reference categories using the relevant statistical difference criterion for each of the analyzed primary indexes in each personal set of indexes; then, subjecting the obtained values reflecting the discriminant ability of each primary index to factor analysis separately for amplitude and time indexes and calculating the values of obtained factors; and, following this, combining the respondents into groups based on that which of factors had the maximum value;
at the second processing stage, obtaining estimation of text, audio, and video stimuli using a criteria system obtained at the previous stage, wherein the second stage comprises the steps of:
a) calculating differences between the stimuli of different categories for each primary index,
b) then, based on these values and the factor coefficients obtained at the first stage, calculating the values of factors which grouping the primary indexes,
c) for each stimulus in a space of primary indexes with the use of discriminant functions calculating the degree of class membership of one of the reference estimations in each group formed at the first stage,
d) weighing the obtained classification results using the value of factor by which this group has been built on the basis of results of item b,
e) summing up the weighed classification results for each group of indexes.
